(19) United States
(12) Patent Application Publication
(10) Pub. No.: US 2010/0215633 A1
(43) Pub. Date: Aug. 26, 2010

(54) Method for feeding dairy cows and dairy goats supplements to increase the C2 hxroylated estrogen (C2) to C16 hydroxlated estrogen (C16) ratio secreted in their urine

(76) Inventor: Robert D. Bibb, Mytle Beach, SC (US)

Correspondence Address: Robert D. Bibb, M.D., 7554 Siena Blvd., Myrtle Beach, SC, 29572, US

(21) Appl. No.: 12/378,758

(22) Filed: Feb. 20, 2009

(51) Int. Cl. A61K 38/44; A61K 36/48; A61K 31/352; A61K 36/87; A61K 31/404; A61P 35/00

(52) U.S. Cl. 424/94.4; 424/757; 514/456; 424/766; 514/415

(57) ABSTRACT

A feed supplement for dairy cows and goats containing various combinations of whole soy, muscadine grapes, Kudzu leaves, brassica group vegetable and or their chemical constituents designed to shift estrogen metabolism towards favorable estrogen chemistry.

### FIELD OF THE INVENTION

The present invention relates generally to adding certain extracts obtained from soy seeds, grape seeds, grape husks and Kudzu leaves to the feed of dairy cows and goats. The invention also includes any and all member of the brassica genus such as broccoli, cabbage, bok choy cauliflower etc. capable of providing the biochemical indole-3-carbinol either as a whole plant or the biochemical extract thereof. The present invention also includes the whole soy seed, the whole dried grape and the whole Kudzu leaf as included as possible additives to the feed of dairy cows and goats.

It can generally be appreciated that in Western societies, there is a high incidence of homionally sensitive cancers such as breast and prostate. Breast and prostate cancers have epidemiologic connections to dairy consumption. Studies in Asia and China where the incidence of these tumors are low suggest a beneficial effect of soy consumption.¹ The soy isoflavones thought to result in this benefit include genistein and daidzein.² Xu and fellow researchers have suggested that the beneficial effect of soy may be the result of a shift in estrogen metabolism toward a more favorable C2 to C16 estrogen ratio.³ Increasing the consumption of vegetables from the brassica genus can also increase the C2 to C16 ratio as measured in human urine. ¹Vincent A. et al. Soy isoflavones: are they useful in menopause? Mayo Clin Proc 2000;75(11):1174-84.²Ibid.³Xu X et al. Effects of soy isoflavones on estrogen metabolism in premenopausal women. Cancer Epidemiol Biomarkers Prev. 2000;9(8):781-6.⁴Fowke J H. Et al. Brassica vegetable consumption shifts estrogen metabolism in healthy postmenopausal women. Cancer Epidemiol Biomarkers Prev 2000;9(8):773-9.

It is now known that the metabolism of estrogens within the body of mammals can result in both “good (C2) and “bad” (C16, C4) estrogens. The effects of estrogen depend on how ingested estrogen from food sources and estrogens endogenously produced within the body are metabolized and what estrogen receptors that they bind to. Risbridger et al have eloquently shown how differentially binding of certain estrogens to estrogen receptor alpha as opposed to estrogen receptor beta can stimulate prostate growth.⁵

Kabat G. et al. measured the urinary C2 (2-hydroxyestrone) to C16 (16 alpha-hydroxyestrone) ratio in postmenopausal women and found a significant correlation of higher breast cancer incidence with higher urinary levels of the C16 estrogen.⁶ Muti and fellow researchers reported on 10,786 women who were followed for an average of 5.5 years and found that among premenopausal women that a higher C2 to C16 estrogen ratio at baseline was associated with a reduced risk of breast cancer.⁷ Muti et al found a similar decreased risk for prostate cancer when the C2 to C16 ratios were high when measured in the urine of men studied for this disease. It becomes clear then that a method to develop a favorable C2 to C16 ratio would decrease the chance of developing breast and prostate cancer.

Lee and fellow researchers have characterized the enzymes responsible for the metabolism of estrogens. Among the most potent enzyme responsible for this function is ⁵Estrogen action on the prostate gland: a critical mix of endocrine and paracrine signaling. Journal of Molecular Endocrinology. September 2007;39(3): 183-188.⁶Kabat G C. et al. Urinary estrogen metabolites and breast cancer: a case-control study. Cancer Epidemiol Biomarker Prev. 1997;413-9.⁷Muti P. et al. Estrogen metabolism and risk of breast cancer: a prospective study of the 2:16 alpha-hydroxyestrone ration in premenopausal and postmenopausal women. Epidemiology 2000; 11(6):635-40.

an inducible chemical known as CYP1A1.⁸ This enzyme is a member of the p450 system.

One of the major mammalian estrogens is 170-estradiol. It is both potent and ubiquitous. The major metabolite of 17β-estradiol is C2 (2-hydroxestrone).⁹

Induction of the CYP1A1 enzyme becomes the object of this patent. If humans were to consume cow's milk and goat's milk or the dairy products made from said milks and if said milks were to contain less C16 and more C2 than the rates of breast and prostate cancer in Western societies could be reduced.

One of the most potent inducers of CYP1A1 are the poly-aromatic hydrocarbons (PAH) produced when meat is charbroiled. Unfortunately the PAH are converted by the CYP1A1 to more carcinogenic compounds. The CYP1A1 is induced by attachment of the PAH to a receptor which then causes the enzyme induction. It turns out the one of the important chemicals in the brassica group is a chemical known as indole-3-carbinol. This chemical attaches to the same PAH receptor, inducing CYP1A1 in a non carcinogen producing fashion.¹⁰ Indole-3-carbinol is the CYP1A1 inducing chemical in the brassica group of plants that will be used as part of this patent.

Xu and colleagues have demonstrated that the isoflavones in soy alter the alter the metabolism of estrogens in a more favorable fashion in premenopausal women.¹¹ Xu has shown a similar benefit in postmenopausal women.¹² Daidzein ⁸Lee A J. Et al. Characterization of the oxidative metabolites of 17beta-estradiol and estrone formed by 15 selectively expressed human cytochrome p450 isofroms. Endocrinology August 2003;144(8):3382-98.⁹Lee A J. et al. Characterization of the oxidative metabolites of 17beta-estradiol and estrone formed by 15 selectively expressed human cytochrome p450 isofroms. Endocrinology August 2003;144(8):3382-98.¹⁰Chen I et al. Indole-3-carbinol and diindolylmethane as ary hydrocarbon receptor agonists and antagonists in T47D human breast cancer cells. Biochem Pharmacol Apr. 26, 1996;51(8)1069-76.¹¹Xu X et al. Effects of soy isoflavones on estrogen metabolism in premenopausal women. Cancer Epidemiol Biomarkers Prev. 1998;7(12):1101-8.¹²Xu X. et al. Soy consumption alters endogenous estrogen metabolism in postmenopausal women. Cancer Epidemiol Biomarkers Prev. 2000;9(8):7816.

and genistein as well as whole soy beans will be used as part of this patent.

Other isoflavones have been found in Kudzu (Pueraria lobata) and one specific isoflavone known as puerarin has been demonstrated to induce CYP1A1 allowing conversion to more favorable estrogens.¹³ Kudzu (Pueraria lobata) and puerarin will be used as part of this patent.

Resveratrol is a potent chemical found in red grapes. The levels of this chemical are thought to be the highest in muscadine varietals. Aluru and Vijayan have demonstrated that low dose reseveratrol can stimulate CYP1A1 in animal studies.¹⁴ Muscadine seed and muscadine husk extract as well as dried muscadine grapes will be used as part of this patent.

### SUMMARY OF THE PATENT

The invention will consist of supplemental feed for dairy cows and dairy goats consisting of various combinations of whole soy beans, dried muscadine grapes, Kudzu leaves and members of the brassica family and or the active ingredients of these plants including resveratrol, daidzein, genistein, puerarin and indole-3-carbinol but not excluding other active ingredients that may be extracted from these plants. The invention will consist of any and all varieties of soy beans, muscadine grapes, Kudzu and brassica varieties found to be effective in producing the end result of favorably altered C2 to C16 ratios as measured by urine analysis of cow's and goat's milk. ¹³Guerra M C. et al. Comparison between Chinese medical herb Pueraria lobata crude extract and its main isoflavone puerarin antioxidant properties and effects on rat liver CYP-cataly-sed drug metabolism. Life Sci 2000;67(24):2997-3006.¹⁴Aluru N. et al. Resveratrol affects CYP1A expression in rainbow trout hepatocytes. Aquat Toxicol 2006 May 10;77(3):291-7.

### DETAILED DESCRIPTION OF THE PREFERRED EMBODIMENT

The present invention is a new dairy cow and dairy goat feed supplement designed to increase the production of C2 (2-hydroxyestrone) and reduce the production of C16 (16-alpha hydroxyestrone) in the milk of dairy cows and goats. With respect to the above description, it is to be realized that the optimum quantity of each of the respective ingredients incorporated into the feed will vary according to the variety of plant chosen. And; therefore, the foregoing description should be illustrative of the principles of the invention. Further, since numerous modifications and changes will occur in the exact composition of the feed supplement, it is not desired to limit the invention to an exact description of the quantities of the ingredients to be incorporated. All suitable modifications and equivalents that may be resorted to should be included in the scope of this invention.

What is claimed is:

1. That certain plants and/or their parts and their chemical constituents induce the p450 enzyme known as CYP1A1.

2. That whole soy beans and their chemical inducers called isoflavones which known as genistein or daidzein induce the CYP1A1 enzyme.

3. That muscadine grapes or their chemical constituent resveratrol induces the enzyme CYP1A1.

4. That Kudzu leaves or the chemical known as puerarin in Kudzu leaves induces the enzyme CYP1A1.

5. That the chemical in the brassica family of vegetable known as indole-3-carbinol induces the CYP1A1 enzyme.

6. That human induction of CYP1A1 can shift estrogen metabolism to produce metabolic conversion of estrogens that are associated with a decrease risk of prostate and breast cancer.

7. That the measure of metabolic shift to favorable estrogens can be measured in the urine of mammals as a ratio of C2 (2-hydroxylestrone) to the C16 (16 alpha-hydroxyestrone)

8. That I have invented a new supplement for feed for dairy cows or dairy goats that includes various combinations of soy beans, muscadine grapes, Kudzu or brassica group vegetables and/or their constituent chemicals genistein, daidzein, resveratrol, puerarin or indole-3-carbinol.

9. That said supplement as outlined in claim 8 is designed to induce CYP1A1 in dairy cows and dairy goats.
